(19) United States
(12) Patent Application Publication
(10) Pub. No.: US 2003/0180291 A1
(43) Pub. Date: Sep. 25, 2003

(54) Method of inactivating viruses

(76) Inventors: Johnny Van Steveninck, Oegstgeest (NL); Alfred L. Van Steveninck, Soesterberg (NL); Johanna Caroline Van Steveninck, Neerloon (NL); Thomas M. A. R. Dubbelman, Alphen A/D Rijn (NL); Johannes W. M. Lagerberg, Leiden (NL); Agatha Brand, Amsterdam (NL); Arthur J. Verhoeven, Amsterdam (NL)

Correspondence Address: KINNEY & LANGE, P.A., THE KINNEY & LANGE BUILDING, 312 SOUTH THIRD STREET, MINNEAPOLIS, MN, 55415-1002, US

(21) Appl. No.: 10/373,422

(22) Filed: Feb. 24, 2003

Related U.S. Application Data
(63) Continuation of application No. 09/720,632, filed on Aug. 14, 2001.
Application No. 09/720,632 is a 371 of international application No. PCT/NL99/00387, filed on Jun. 23, 1999.

(30) Foreign Application Priority Data
Jun. 23, 1998 (NL) 1,009,472

(51) Int. Cl. A61K 39/395; A61K 31/519; A61K 31/675; C12Q 1/68; A01N 57/00; A61K 39/00; A01N 43/90

(52) U.S. Cl. 424/140.1; 514/89; 514/262.1; 435/6

(57) ABSTRACT

The invention relates to a method of inactivating viruses in an erythrocytes-containing liquid with the use of light, wherein a sensitizer is added to the liquid. Further, an erythrocyte-binding agent is added to the liquid, which protects against the influence of singlet oxygen and/or radicals.

The present invention relates to a method of inactivating viruses in an erythrocytes-containing liquid with the use of light, wherein a sensitizer is added to the liquid.

Such a method is disclosed in the Dutch patent application 1006219 (not pre-published). In this method a sensitizer is added to an erythrocytes-containing liquid such as, for example, diluted blood, after which the liquid is irradiated with light, thereby exciting the sensitizer. Interaction between the excited sensitizer and oxygen present results in reactive oxygen species causing a virus present to become inactivated. Other interactions of the excited sensitizer may result in the occurrence of radicals and other reactive species, which may also cause a virus present to become inactivated.

A disadvantage of applying such a known method of inactivating viruses is that apart from the virus, the erythrocytes themselves are damaged by reactive oxygen species. This limits the extent to which the viruses can be inactivated.

It is the object of the present invention to avoid this disadvantage. According to the present invention this object is realized by adding to the liquid an erythrocyte-binding agent, which protects against the influence of singlet oxygen and/or radicals. The liquid will generally contain oxygen.

As can be seen from the Examples below, the addition of the agent according to the invention provides the erythrocyte with a greater degree of protection against photodynamic damage than the virus particle.

According to a preferred embodiment, the agent is bound to a carrier binding to the erythrocyte's surface, preferably covalently. This means that more agent molecules are available. Moreover, the specificity may be chosen freely. Preferably, said carrier is an antibody against a surface antigen of the erythrocyte, such as IgM. IgM binds weakly so that it can later be easily removed again.

In accordance with another feature of the invention the agent is a ligand for a transport protein, preferably an anion transport protein.

Such transport proteins occur in relatively large numbers on the cell surface, so that they conveniently provide for the possibility of protecting the cell surface.

In accordance with another feature of the invention the transport protein is a Band 3-protein forming part of a complex that, among other things, provides for HCO₃⁻ transport that is important for erythrocytes. The “concentration” of Band 3-proteins at the cell surface is high (˜10⁶ copies per cell). Apart from that, no/virtually no Band 3-related structures occur on the surface of membrane virus particles, so that Band 3-specific ligands will have a much lower affinity for the surface of the virus particle. Moreover, Band 3 has been studied extensively and there are many ligands known that bind to Band 3.

According to one embodiment, the ligand binds to a transport site of the transport protein, the ligand preferably being pyridoxal phosphate (PDP) or a derivative thereof. PDP is a vitamin, so that its retention in treated erythrocyte suspension in relatively small amounts will pose no problems. It should be noted that when PDP (which is itself also a sensitizer) is used, the wave-length of the light used must be chosen such that only the compound added and intended as sensitizer will be excited, and not PDP.

According to another embodiment the ligand binds to the protein's channel domain, the ligand preferably being dipyridamol (PIP) or a derivative thereof. DIP is a registered drug, the use of which is known to cause limited side effects.

According to yet another embodiment the ligand binds to the protein's conformation site. Such a ligand may, for example, be a translocation inhibitor such as, for example, nifluminic acid.

The invention will now be elucidated with reference to the exemplary embodiments.

### EXAMPLE 1

Shortly after blood samples were taken from healthy donors, the blood was centrifuged, and the erythrocytes were washed three times with NaCl-HEPES buffer (150 mM NaCl-10 mM HEPES (ICN Biochemicals, Cleveland Ohio, USA) pH 7.6). Dilution in this buffer provided a 2% erythrocyte suspension. 10⁵ Vesicular Stomatitis virus (VSV) particles per ml were added (San Juan strain, obtained from the Department of Virology, LUMC, Leiden). Further, dimethyl-methylene blue (Aldrich, the Netherlands) was added as sensitizer, in a final concentration of 1 μM. The erythrocyte suspension obtained was divided into four portions (A, B, C and D). Nothing was added to portion A; to portion B 1 mM pyridoxal phosphate PDP was added; to C 0.1 mM dipyridamol DIP; and to D 1 mM pyridoxal phosphate plus 0.1 mM dipyridamol. Subsequently the portions A, B, C and D were irradiated with light from a 500 W halogen lamp, which light was passed through a water filter (to eliminate heat effects) and then through a low-pass filter having a cut-off value of 590 nm. The final light intensity was 15 mW/cm². The following factors were established:

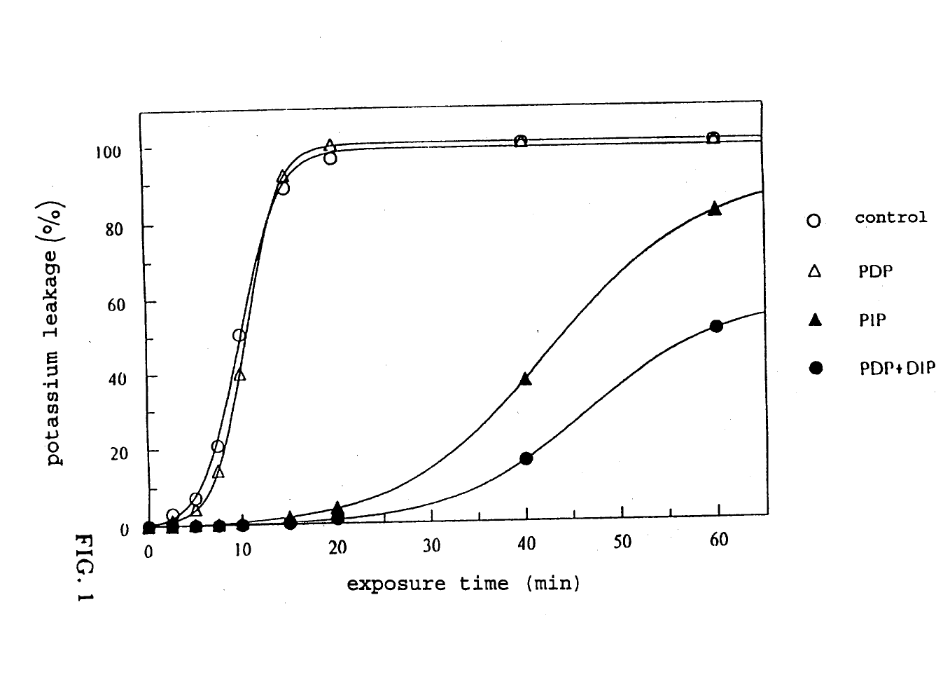

potassium leakageage (K leakage) from erythrocytes after different exposure times (FIG. 1);

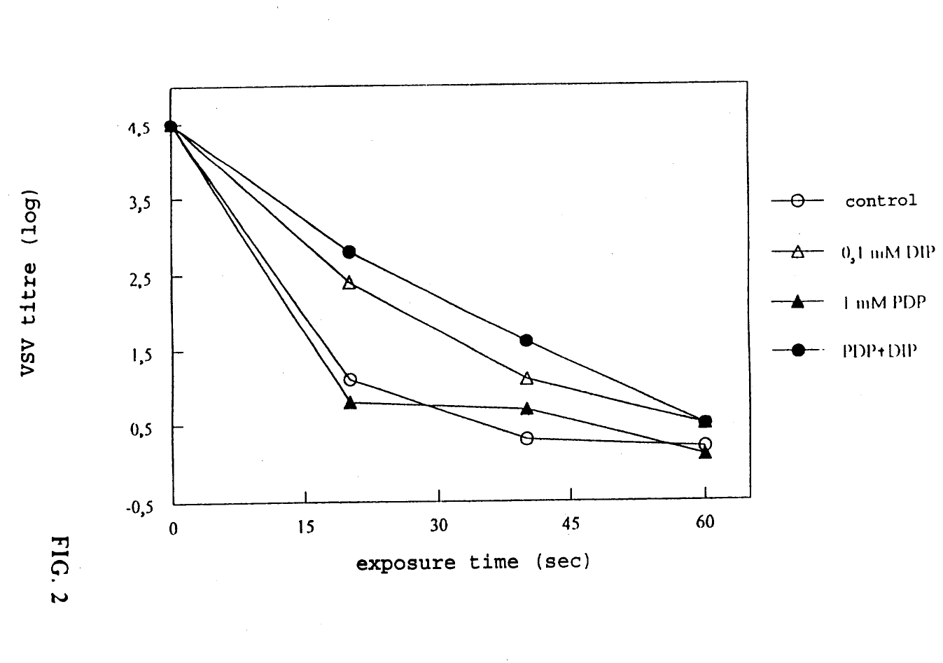

virus inactivation after different exposure times (FIG. 2).

The combination of pyridoxal phosphate/dipyridamol was shown to provide the virus with little protection against photodynamic inactivation, while (particularly after short exposure times) the erythrocyte was well protected against (photodynamically caused) cell wall damage, as appears from the small K-leakage. A large K-leakage signifies that considerable cell damage has been incurred.

### EXAMPLE 2

A control experiment was carried out to show that light exposure of the erythrocyte suspension without the sensitizer does not cause any photodynamic damage to the erythrocyte and/or the virus.

The experiment of Example 1 was repeated, this time in the presence of dipyridamol and/or pyridoxal phosphate, but in the absence of the sensitizer dimethyl-methylene blue. Light exposure of the erythrocyte suspension did indeed have no effect at all with regard to the virus inactivation (results not shown).

### EXAMPLE 3

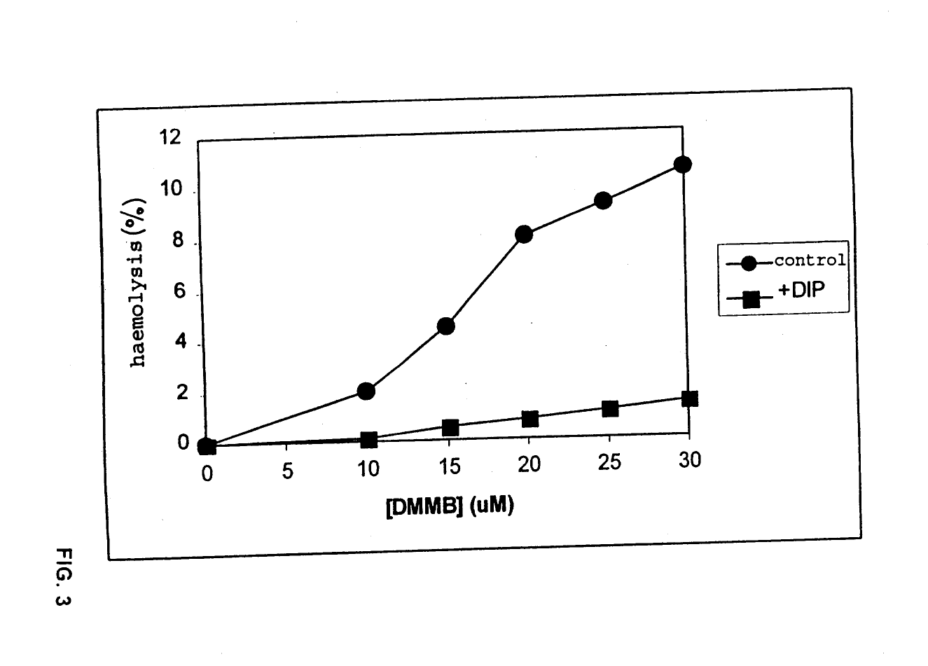

The possible interference of erythrocyte ligands with the efficiency of virus inactivation was also studied in more concentrated erythrocyte suspensions (30% haematocrit). For the photodynamic treatment dimethyl-methylene blue (DMMB), red light (as in Example 1) and dipyridamol as erythrocyte ligand and scavenger. In these experiments the minimal DMMB concentration was determined, that was necessary to effectuate at an exposure time of 2 minutes, a more than ³log virus titre reduction of Vesicular Stomatitis (VSV, strain Indiana, obtained from the Department of Biotechnology CLB, Leiden, the Netherlands), PseudoRabies virus (PSR, strain Bartha K61, obtained from Duphar, Weesp) and Humane Immunodeficiency Virus Type 1 (HIV-1, strain HTLV-IIIb, obtained from the National Cancer Institute, Bethesda, United States of America). To this end 10⁵ virus particles per ml (for VSV and PSR) or 10⁶ virus particles per ml (for HIV-1) were incubated for 5 minutes with humane erythrocytes (30% Ht, suspended in 150 mM NaCl, 50 mM glucose, 30 mM mannitol and 1.2 mM adenine), after which dipyridamol was added to half the incubations (final concentration 0.1 mM). After a further 10 minutes incubation at room temperature, increasing amounts (0-16 μM) of DMMB were added to the incubations which were then incubated in the dark for 6 minutes at room temperature before exposing the suspensions for 2 minutes to light. The addition of dipyridamol (0.1 mM) did not, or only barely appear to affect the effective concentration of sensitizer (Table I, below) and consequently did not appear to decrease the efficiency of virus inactivation The addition of dipyridamol did, however, result in effective protection of the erythrocytes, which was expressed, among other things, by a very strong reduction of the haemolysis measured after the erythrocytes had been stored for 12 days at 4° C. FIG. 3 shows the results of haemolysis measurements 12 days after the photodynamic treatment (2 minutes light exposure and increasing DMMB concentrations).

TABLE 1

effective [DMMB] (μM)

| virus | control | +DIP |
|---|---|---|
| VSV | 4 | 5 |
| PSR | 4 | 4 |
| HIV-1 | 8 | 8 |

1. A method of inactivating viruses in an erythrocytes-containing liquid with the use of light, wherein a sensitizer is added to the liquid, characterized in that to the liquid an erythrocyte-binding agent is added, which protects against the influence of singlet oxygen and/or radicals.

2. A method according to claim 1, characterized in that the agent is bound to a carrier binding to the erythrocyte's surface.

3. A method according to claim 2, characterized in that the carrier is an antibody against a surface antigen of the erythrocyte.

4. A method according to one of the preceding claims, characterized in that the agent is a ligand for a transport protein.

5. A method according to claim 4, characterized in that the transport protein is an anion transport protein.

6. A method according to claim 4 or 5, characterized in that the transport protein is a Band 3-protein.

7. A method according to one of the preceding claims, characterized in that the ligand binds to a transport site of the protein.

8. A method according to claim 7, characterized in that the ligand is pyridoxal phosphate (PDP) or a derivative thereof.

9. A method according to one of the preceding claims, characterized in that the ligand binds to the protein's channel domain.

10. A method according to claim 9, characterized in that the ligand is dipyridamol (PIP) or a derivative thereof.

11. A method according to one of the preceding claims, characterized in that the ligand binds to the protein's conformation site.

12. A method according to claim 11, characterized in that the ligand is nifluminic acid.
